(19) United States
(12) Patent Application Publication
(10) Pub. No.: US 2009/0254095 A1
(43) Pub. Date: Oct. 8, 2009

(54) SUBCUTANEOUS TUNNELING DEVICE

(76) Inventors: Blake Levine, Evanston, IL (US); Louise Giam, Chicago, IL (US); Christa Nelson, Chicago, IL (US); Varun Kshettry, Chicago, IL (US)

Correspondence Address: WHYTE HIRSCHBOECK DUDEK S.C.;INTELLECTUAL PROPERTY DEPARTMENT, 33 East Main Street, Suite 300, Madison, WI, 53703-4655, US

(21) Appl. No.: 12/406,824

(22) Filed: Mar. 18, 2009

Related U.S. Application Data
(60) Provisional application No. 61/037,502, filed on Mar. 18, 2008.

(51) Int. Cl. A61B 17/34; A61B 17/00

(52) U.S. Cl. 606/108; 606/129; 606/185

(57) ABSTRACT

A subcutaneous tunneling device is provided with an adjustable handle and a user actuated penetrating mechanism for greater control and ease of use during a tunneling procedure.

### CROSS-REFERENCE TO RELATED APPLICATIONS

This application claims the benefit of U.S. Provisional Application Ser. No. 61/037,502 filed on Mar. 18, 2008 and titled Tunneling Tool with Penetrating Device and Adjustable Handle, which is hereby incorporated by reference in its entirety herein.

### FIELD OF THE INVENTION

Various embodiments of the present invention relate to subcutaneous tunneling tools and methods of use. More particularly various embodiments of the present invention relate to subcutaneous tunneling tools employing an improved tip and handle mechanisms, and methods of using the same.

### BACKGROUND OF THE INVENTION

Tunneling is a surgical procedure to create a pathway under the skin for passing a wire, catheter, or shunt from one area to another. It is commonly used in four types of procedures: Deep Brain Stimulation (DBS), Cortical Stimulation (CS), Spinal Cord Stimulators (SCS), and Ventriculoperitoneal Shunt (VPS) placements. In DBS and CS, wires are needed under the skin to connect electrodes implanted in the brain to an internal pulse generator and power supply placed just below the clavicle. DBS is currently FDA-approved to treat movement disorders such as Parkinson's disease, essential tremor, and dystonia. Current research shows promise for many more indications such as depression, obsessive-compulsive disorder, epilepsy, and obesity. CS is similar to DBS, but the target sites of stimulation are areas on the surface of the brain. CS is currently FDA-approved to treat chronic pain. VPS is a procedure in which a catheter is placed within the fluid filled ventricles within the brain and connected to a catheter that is tunneled under the skin down to the peritoneal cavity in order to drain cerebrospinal fluid (CSF).

In order to create a subcutaneous (under the skin) tunnel for the lead, a tunneling device is needed. These devices usually consist of a malleable metal tube with a bullet-shaped tip on one end, and a handle on the other end. The tunneling begins under the scalp at the apex of the head, and continues down the side of the head, behind the ear, down the neck to an area just below the clavicle. Here, in the case of neurostimulator placements, an incision is made to place the implantable pulse generator (IPG) and connect it to a lead that can be threaded through the tunneling device back up to the scalp electrode.

Neurosurgeons are often frustrated with current tunneling devices for several reasons, including: 1) Force requirements change during tunneling: The current tunneling devices often have relatively blunt tips. This is because a sharper tip can be dangerous when tunneling near vessels and nerves in the neck that supply the head and arm. However, there are areas with dense tissue where a blunt tip makes tunneling difficult due to the brute force required and loss of control. The densest tissue is encountered when tunneling directly behind the ear. Here, a sharper tip is ideally suited to help cut through fascia without requiring excessive force. 2) Tunneling tools are often difficult to control: Long malleable tools are difficult to control because the handles are often at the opposite end of the tip of the tool. Applying force at a distance from the tip diminishes control over the tip as one is trying to direct the tip under the skin. Additionally, it can create bending forces in the malleable metal, which distorts the shape of the tunneling device and decreases force transmitted into the tip. The use of a tool that allows force to be applied as close to the tip as possible can improve the surgeon's control.

Various configurations of subcutaneous tunneling devices have been previously disclosed. By example, an integrated subcutaneous tunneling and carrying tool is provided in U.S. Pat. No. 6,605,094, hereby incorporated by reference in its entirety herein. Yet another example provides a subcutaneous tunneling instrument and method in U.S. Pat. No. 4,832,687, hereby incorporated by reference in its entirety herein, and in particular columns 1-4 are hereby incorporated by reference. An additional example, U.S. Pat. No. 7,018,384 provides a medical passing device and method, incorporated by reference in its entirety herein, and in particular columns 1-8 are hereby incorporated by reference. By example, U.S. Pat. No. 6,666,846 provides a medical device introducer and obturator and methods of use, which is hereby incorporated by reference in its entirety herein, and in particular columns 1 and 3-9 are hereby incorporated by reference.

These flaws with current tools not only make the surgery more frustrating for the surgeon, but can also lead to complications. Previously known tunneling tools have presented several issues to neurosurgeons during tunneling procedures. By example, 1) Operator fatigue: Brute force needed to push the tool through often thick fascia combined with poor device ergonomics has often led to operator fatigue; 2) Increased risk of injury to vital structures due to poor maneuverability and excessive force, which can lead to an increased risk of damage to important arteries, veins, and nerves. There has also been an increased risk of accidentally tearing through the skin as one applies a significant amount of force load to skin; and 3) Additional incisions are often necessary near the latter half of a tunnel tract, as the surgeon often can no longer adequately steer the device under the skin. As a result, the surgeon will often make an intermediate incision at the point where the tip can no longer be advanced and reinsert the tunneling device at the new incision to continue the tract. This can occur in approximately 50% of tunneling cases for various procedures. A second incision carries with it the need to shave hair in a second location, a second wound site, and most importantly, a greater risk of wound and hardware infection. Hardware infection is an extremely serious complication, often resulting in long hospital stays (average length of stay is about ˜14 days), intravenous antibiotics, and the need for additional surgeries to remove and replace hardware. This results in increased morbidity and mortality for patients and increased costs for hospitals (additional costs ˜$30,000 without need for surgery, ˜$45,000 with additional surgery). Due to the government no longer reimbursing surgery-related infections, this cost burden will now be placed on hospitals. It would be advantageous to have a tunneling device that allows greater control during tunneling and can reduce the need for creating second incisions.

### SUMMARY OF THE INVENTION

Briefly in one aspect, a tunneling device for subcutaneously placing a medical tube is provided. The tunneling device includes an elongated tubular member, an adjustable handle mechanism in communication with the tubular member, and a tunneling mechanism comprising a blade member, a blade housing, and a blade actuating mechanism.

In accordance with an alternative embodiment, a method for creating a tunnel under the skin during surgery is provided, comprising the steps of: Using a tunneling device compromised of an elongated tubular member, tip with an orifice at its apex housing an inner penetrating device and an actuating mechanism.

### BRIEF DESCRIPTION OF THE FIGURES

FIG. 1 is a perspective view of a subcutaneous tunneling device in accordance with at least one embodiment of the present invention;

FIG. 2 is a perspective view of the distal end of the tunneling device in accordance with at least one embodiment of the present invention;

FIG. 3 is a perspective view of the device according to FIG. 2 in the blade open position;

FIG. 4 is a side view of a subcutaneous tunneling device in accordance with at least one embodiment of the present invention;

FIG. 5 is a side view of the device according to FIG. 4 with the blade member extended;

FIG. 6 is a side view of the device according to FIGS. 4 and 5 with the blade actuating member removed from the tubular member.

### DETAILED DESCRIPTION OF VARIOUS EMBODIMENTS

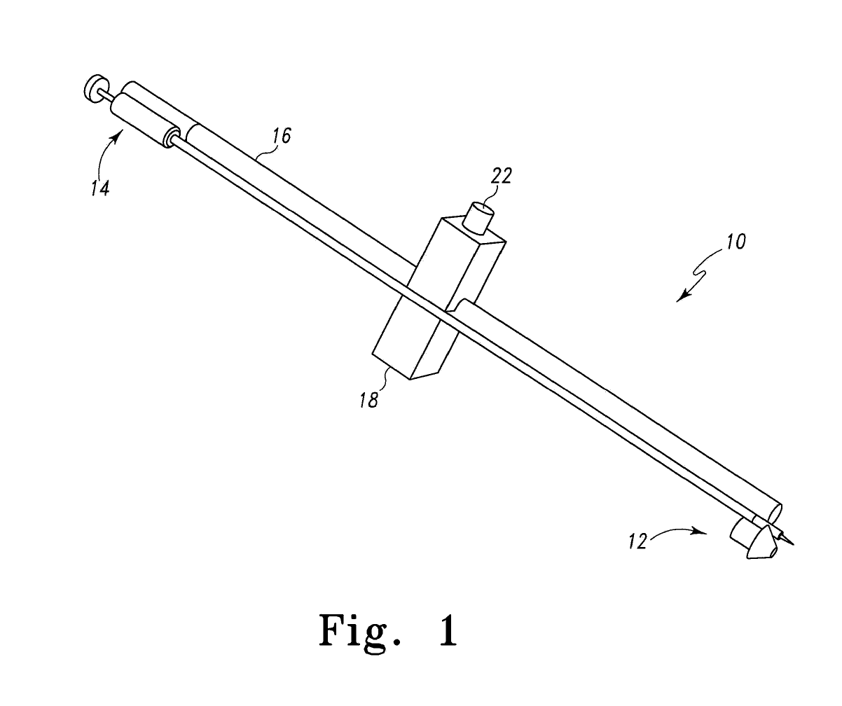

Referring to FIG. 1, a subcutaneous tunneling device 10 is provided. The device 10 has a distal end 12, a proximal end 14, a tubular member 16 and handle 18. The device 10 is used for passing an elongated medical tube 16 subcutaneously under the skin of a human or animal subject for the purpose of creating a tunnel under the skin through which medical leads and catheters can be passed. The tubular member 16 has a proximal end and a distal end, the tubular member 16 defining a channel between the proximal end 14 and the distal end 12. The tubular member 16 has a lumen for which a medical lead, electrode, catheter, or shunt can be passed through. The purpose of this device 10 is to facilitate the tunneling process. The device 10 has a handle 18 that locks onto the tubular member 16 and can slide along the length of the tube 16 to allow control of the tunneling device 10 at variable points along the tubular member 16.

The handle member 18 can be formed in a plurality of ergonomically shaped configurations. Alternatively, it is contemplated that the handle 18 can be customized for a particular health care worker, such as a neurosurgeon, based upon their liking. In at least one embodiment, the tubular member 16 is made of a malleable and/or bendable metal or alternately suitable material.

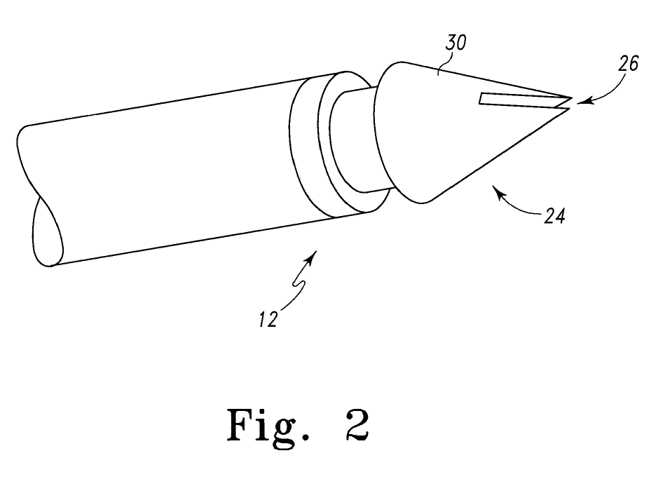

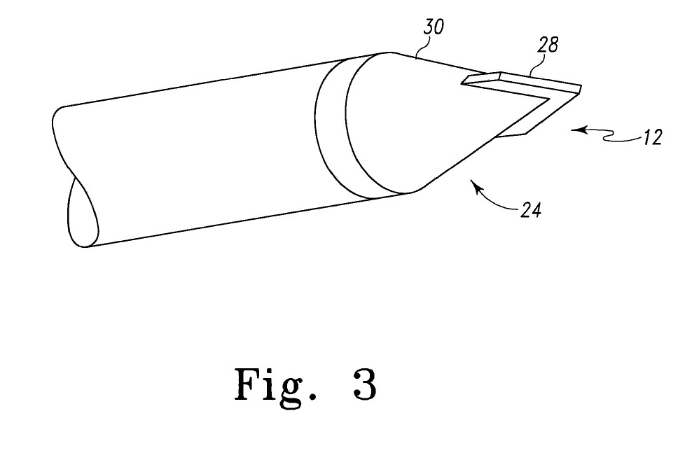

Referring to FIGS. 2-3, the distal end 12 of the device 10 is provided. The distal end 12 includes a blade housing 30 which has a tip 24 with an orifice 26 at its apex. Within the blade housing 30 is a penetrating device 28, such as a blade, scalpel or other suitable cutting instrument. In the present embodiment, the device 10 has a fixed blade 28 covered by a retractable blade housing 30. To cover the blade 28 there is a switch that is actuated by the operator of the device 10, which allows the blade housing 30 to extend over and substantially cover the blade 28. The tip 24 can be conical, triangular, or an alternative shape suitable for penetrating. Alternatively, the present embodiment can be modified such that the blade housing 30 is stationary and the blade 28 is repositionable.

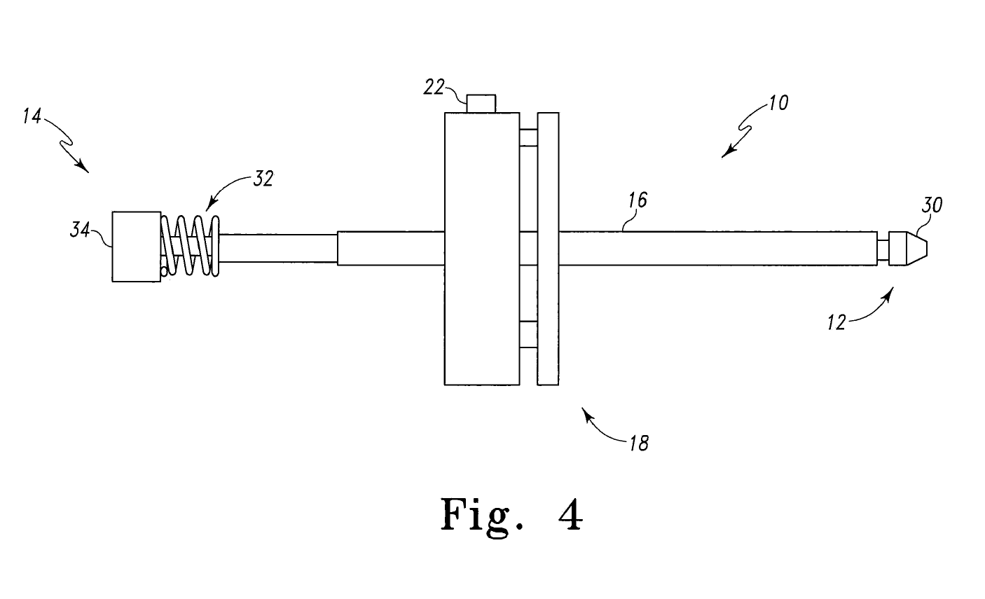

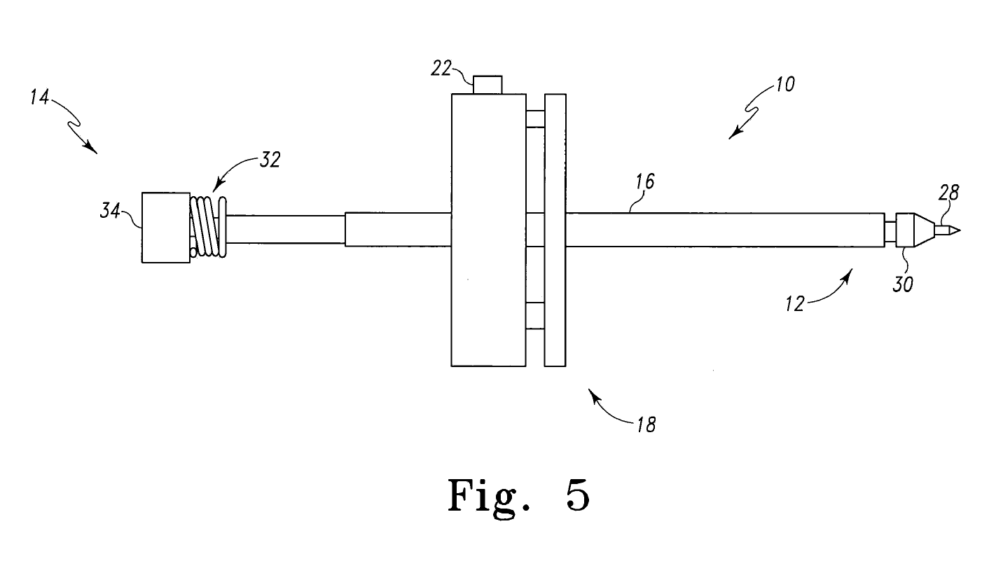

Referring to FIGS. 4-5, an alternative embodiment of the present invention is provided. The device 10 includes a retractable blade 28 covered by a sheath 30. The blade is actuated by a spring 32 loaded button 34 at the proximal end 14 of the device 10. The handle member 18 has an opening to receive the tubular member 16 and an adjusting mechanism 22 for sliding the handle along the length of the tubular member to allow control of the tunneling device 10 at variable points along the tubular member 16. The handle member 18 locks onto the tubular member 16 and contains an actuating mechanism 22, that when activated, releases the handle off a particular position along the tubular member 16 and allows the handle to slide along the tubular member 16. When the actuating mechanism 22 is released it is frictionally locked onto the tubular member 16 at that position. Alternatively, indentations (not shown) are provided at various positions along the tubular member 16. The handle 18 locks into a given indentation along the tubular member 16 and the actuating mechanism 22 is used to release the handle 18 off any particular indentation, thereby allowing the handle to slide to the next indentation on the tubular member to which it will lock in place. The tubular member can have a cross section that is circular, oval, rectangular or an alternative shape.

The device 10 includes a handle actuating mechanism 22 and a connecting mechanism between the inner penetrating device 36 and an actuating mechanism 38 on the tube accessible to the operator; such that when the actuating mechanism 38 is engaged, the penetrating device 28 protrudes out the tip 30; and when the actuating mechanism 38 is engaged again, the penetrating device 28 retreats to within the tip 30. Alternatively, a tip actuating mechanism (not shown) can be placed on the tubular member 16 or in conjunction with the handle mechanism 18, such that as the handle mechanism compresses the tubular member or the actuating mechanism compresses the tubular member 16, the penetrating device 36 is rigidly maintained within the tubular member, thereby aiding use of the device 10. Alternatively, the distance the penetrating device 28 protrudes from the housing 30 can be altered based upon the particular procedure and type of fascia through which the device is tunneling.

The invention utilizes two separate innovations in the handle and the tip to enable surgeons to modulate the sharpness of the tip so that a sharper tip can be used to get through thick dense areas and a duller tip can be used to safely navigate through areas with nerves and blood vessels. Thus, the invention claims both a device and a method. The method claimed utilizes an elongated medical tube in which is sheathed a penetrating device for the purpose of creating a tunnel through the patient's tissue is disclosed. The medical device of the present invention comprises a passer having a proximal end and a distal end, the passer defining a channel between the proximal end and the distal end. First, the device has a handle that latches on and slides along the length of the tubular member to allow control of the tunneling device at variable points along the tubular member. Second, the device has a retractable blade covered by a sheath. To uncover the sheath and utilize the blade, there is a switch that is actuated by the operator of the device.

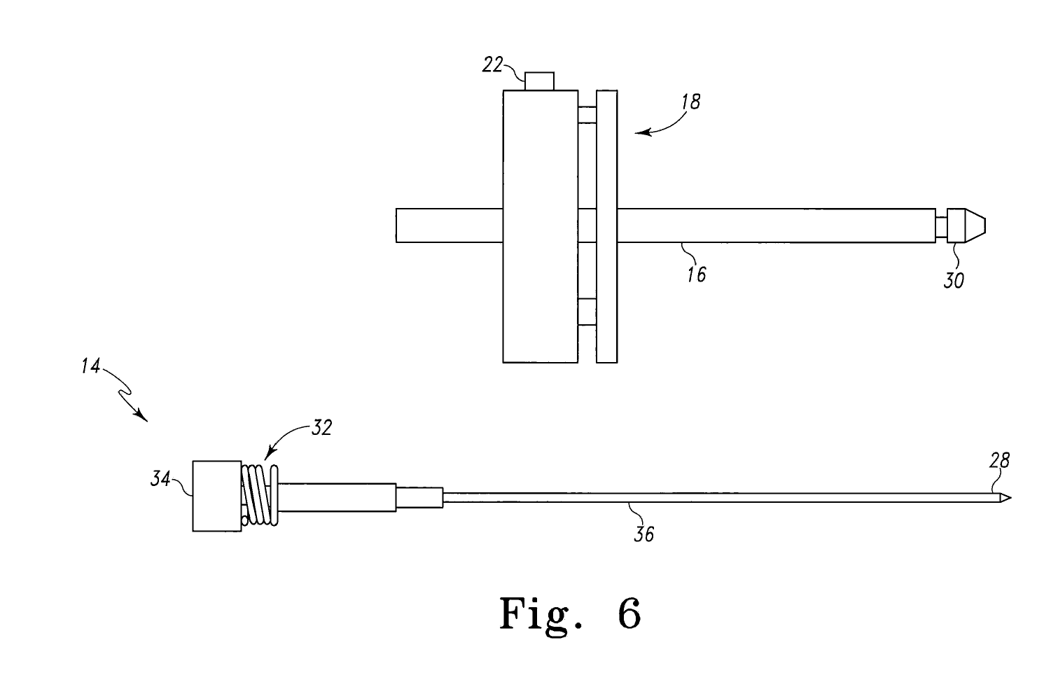

Various embodiments of the present invention are used for creating a tunnel under the skin during a surgical procedure. The steps include using a tunneling device comprised of elongate tubular member and handle as shown in FIGS. 1-6, followed by advancing the tunneling device under the skin and adjusting the handle position along the length of the tubular member for optimal device control.

Additionally, method for creating a tunnel under the skin during surgery includes the use of a tunneling device compromised of an elongate tubular member, tip with an orifice at its apex housing an inner penetrating device and an actuating mechanism 22. The procedure includes advancing the tunneling device under the skin wherein the blade is sheathed by the tip and operator engagement of the actuating mechanism 22 exposes the blade such that it is not sheathed by the tip, allowing the device to be advanced with the blade exposed. The operator can engage the actuating mechanism 22 to retreat penetrating device so that it is sheathed by the tip. Further advancing the tunneling device under the skin and use of the actuating mechanism to expose the blade when desired by the operator is contemplated.

The various embodiments are given by example and the scope of the present invention is not intended to be limited by the examples provided herein. Although the invention has been described in detail with reference to preferred embodiments, variations and modifications exist within the scope and spirit of the invention as described and defined in the following claims.

1. A tunneling device for subcutaneously placing a medical tube comprising:
an elongated tubular member;
an adjustable handle mechanism in communication with the tubular member; and
a tunneling mechanism comprising a blade member, a blade housing, and a blade actuating mechanism.

2. The device according to claim 1, wherein the handle is slidably engaged with the handle along the length of the tubular member to allow control of the tunneling device at variable points along the tubular member.

3. The device according to claim 1, wherein the adjustable handle mechanism locks the handle on the tubular member.

4. The device according to claim 1, wherein upon release of the adjustable handle mechanism the handle is released from the member and freely slides along the tubular member, and when the actuating mechanism is released, locks onto the tubular member at that position.

5. The device according to claim 1, wherein the tubular member has a plurality of indentations for assisting the stationary placement of the handle.

6. The device according to claim 1, wherein the handle has a conformation to allow ergonomic gripping.

7. The device according to claim 1, wherein the tubular member has a lumen for which a medical lead, electrode, catheter, or shunt can be passed through.

8. The device according to claim 1, wherein the blade housing is a tip with an orifice at its apex.

9. The device according to claim 8, with a connecting mechanism between the inner penetrating device and an actuating mechanism on the tube accessible to the operator; such that when the actuating mechanism is engaged, the inner penetrating device protrudes out the tip; and when the actuating mechanism is engaged again, the penetrating device retreats to within the tip.

10. The device according to claim 8, where the housing is conical shaped.

11. The device according to claim 8, where the penetrating device is a sharp uniform point.

12. A method for creating a tunnel under the skin during surgery, comprising the steps of: using a tunneling device comprised of elongate tubular member and handle as stated according to claim 1 advancing the tunneling device under the skin; adjusting the handle position along the length of the tubular member for optimal control.

13. A tunneling device for subcutaneously placing a medical tube comprising:
an elongated tubular member;
an adjustable handle mechanism in communication with the tubular member; and
a tunneling mechanism comprising a blade member, a blade housing, and a blade housing actuating mechanism.

14. A method for creating a tunnel under the skin during surgery, comprising the steps of: Using a tunneling device compromised of an elongate tubular member, tip with an orifice at its apex housing an inner penetrating device and an actuating mechanism as stated according to claim 8.

15. The method according to claim 14 further comprising the step of advancing the tunneling device under the skin wherein the blade is sheathed by the tip.

16. The method according to claim 15 further comprising the steps of actuating a mechanism to expose the blade such that it is not sheathed by the tip and advancing the tunneling device with the blade exposed.

17. The method according to claim 16 further comprising the step of actuating the mechanism to retreat the penetrating device so that it is sheathed by the tip.
